(19) United States
(12) Patent Application Publication
(10) Pub. No.: US 2025/0346566 A1
(43) Pub. Date: Nov. 13, 2025

(54) Process For Preparation of Imeglimin and Salts Thereof

(71) Applicant: HARMAN FINOCHEM LIMITED, Mumbai, Maharashtra (IN)

(72) Inventors: Nitin Sharadchandra Pradhan, Thane (West), Maharashtra (IN); Vijay Trimbak Kadam, Aurangabad, Maharashtra (IN); Sandip Babanrao Pawar, Aurangabad, Maharashtra (IN); Ashitosh Shivaji Jadhav, Ahmednagar, Maharashtra (IN); Harpreet Singh Minhas, Mumbai, Maharashtra (IN); Gurpreet Singh Minhas, Mumbai, Maharashtra (IN)

(21) Appl. No.: 18/860,062

(22) PCT Filed: Apr. 25, 2023
(86) PCT No.: PCT/IN2023/050402

§ 371 Date: Oct. 25, 2024

(30) Foreign Application Priority Data
Apr. 27, 2022 (IN) 202221024674

(51) Int. Cl. C07D 251/10

(52) U.S. Cl. CPC C07D 251/10

(57) ABSTRACT

The present invention discloses an improved process for the preparation of Imeglimin & salts thereof. More particularly, the present invention discloses chiral resolution of Racemic Imeglimin and its salts which is industrially advantageous and economically significant.

### FILED OF THE INVENTION

The present invention relates to an improved process for the preparation of Imeglimin & salts thereof. More particularly, the present invention relates to chiral resolution of Racemic Imeglimin and its salts which is industrially advantageous and economically significant.

### BACKGROUND OF THE INVENTION

Imeglimin is chemically known as (6R)-(+)-4-dimethylamino-2-imino-6-methyl-1,2,5,6-tetrahydro-1,3,5-triazine hydrochloride, having the structure compound of formula (I).

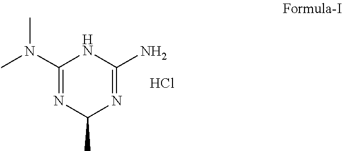

Imeglimin was first discovered by Poxel and developed in partnership with Sumitomo Dainippon Pharma Co. Ltd, launched with the brand name of Twymeeg® in Japan for the treatment of type-2 diabetes mellitus. Imeglimin was first approved in Japan on Jun. 23, 2021 for the treatment of type-2 diabetes mellitus. Imeglimin is under phase-3 clinical trial in USA & Europe.

U.S. Pat. No. 7,034,021B2 first discloses the Imeglimin. Further U.S. Pat. No. 7,034,021B2 does not disclose process for preparation of Imeglimin by any specific example. U.S. Pat. No. 7,501,511B2 (herein after US′511) discloses resolution process for preparation of Imeglimin HCl which comprises reaction of racemic Imeglimin free base with (−)-di-O,O′-p-tolyl-L-tartaric acid in alcohol solvent followed by filtration and recrystallization from DMF/ethanol mixture to obtain the corresponding tartrate salt of compound of formula (VII) in 33% yield having chiral purity of 70% ee. This tartrate salt was dissolved in water/ethyl acetate mixture followed by treatment with hydrochloric acid. The product was recovered from aqueous phase by recrystallization from ethanol to obtain Imeglimin HCl compound of formula (I) as white powder having overall yield 10%. The organic phase was recovered in order to recycle the (−)-di-O,O′-p-tolyl-L-tartaric acid.

The schematic representation of Imeglimin HCl process as disclosed in US′511 is given below in Scheme-I.

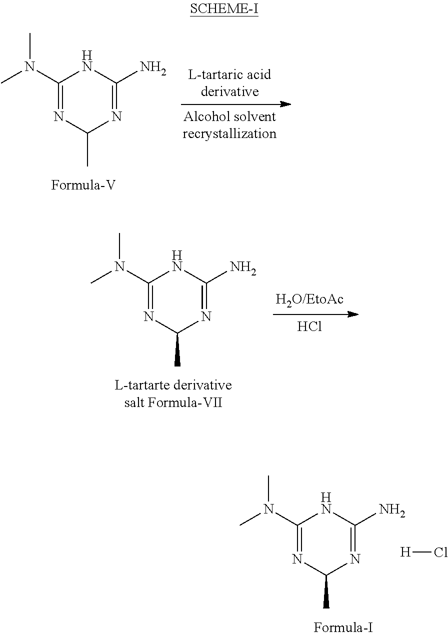

The process as disclosed in US′511 has several disadvantages such as low yield & low chiral purity. Further during the process many impurities generated which were carried forward in the final API, i.e. Imeglimin HCl.

U.S. Pat. No. 8,742,103B2 (herein after US′103) discloses a process for preparation of Imeglimin HCl which process comprises reaction of compound of formula (II) with compound of formula (III) in solvent in presence of PTSA to obtain racemic compound of formula (IV). Further, the racemic compound of formula (IV) reacts with L (+)-Tartaric acid to obtain (+) Imeglimin-L-Tartrate salt in 40-45% yield. Further transformation of the isolated diastereomer of the tartrate salt to HCl salt in 50-55% yield followed by recovery of tartrate salt of (+) Imeglimin from the mother liquor.

The schematic representation of Imeglimin HCl process as disclosed in US'103 is given below in Scheme-II.

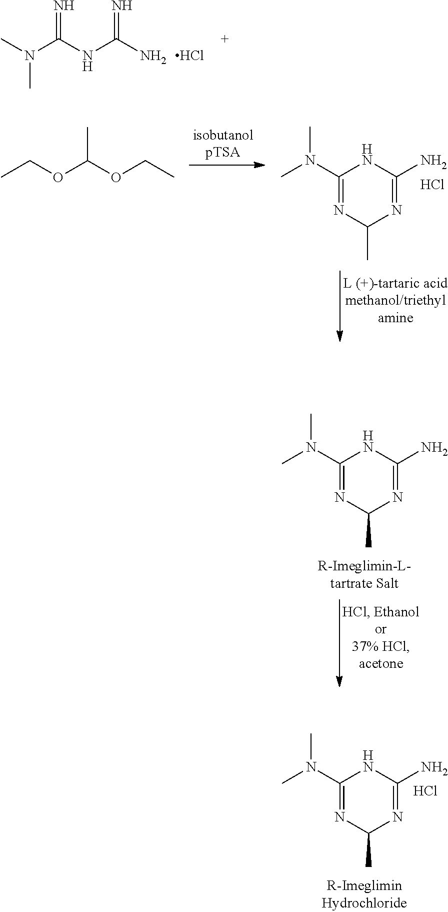

The major disadvantage of Imeglimin HCl process as disclosed in US'103 results in very low yield i.e. 40-45% and further silent about chiral purity of tartrate salt of Imeglimin. One more disadvantage of Imeglimin HCl process as disclosed in US'103 is the yield of final Imeglimin HCl, which is in the range of 50-55% followed by the additional step of recovery of Imeglimin tartrate from the mother liquors which is not suitable for the large-scale Industrial production.

As is evident from the foregoing, all the available prior arts discussed above suffer from many disadvantages such as incomplete reactions, tedious and cumbersome work up procedures, additional step of recovery from the mother liquor, higher temperature condition, longer reaction times, use of excess reagent and solvents which affect the overall yield as well as the quality of the final product.

Therefore, there remains a need in the art for improved process for the preparation of Imeglimin HCl having high chiral purity and high yield which can overcome the drawbacks of the prior arts processes. In pursuit of the above, the present inventors have surprisingly found an efficient process for the preparation of Imeglimin HCl which offer great advantages over the prior art processes in terms of high yield, high chiral purity and less effluents and further simple and scalable procedure suitable for large scale industrial production of Imeglimin HCl.

### OBJECT OF THE INVENTION

The principal objective of the present invention is to provide process for preparation of Imeglimin HCl compound of formula (I) having high yield & high chiral purity by isolating Imeglimin HCl in a single lot and avoiding any recovery from the mother liquor.

Another objective of the present invention is to provide process for preparation of Imeglimin HCl compound of formula (I) via resolution of racemic Imeglimin base compound of formula (III) by treating with a suitable chiral amino acid or its derivative to obtain R-Imeglimin L-amino acid salt of formula (IV) having high yield, more than 99.85% Chiral purity & more than 99.5% HPLC purity, which is upon treatment with HCl to obtain R-Imeglimin HCl salt.

### SUMMARY OF THE INVENTION

In line with the above objective, the present invention provides a process for preparation of Imeglimin HCl compound of formula (I), which process comprises;
a) reacting compound of formula (II)

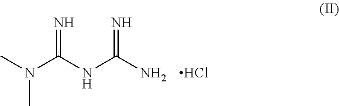

with a compound of formula (III) in a solvent

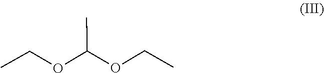

in presence of p-toluene sulphonic acid monohydrate under suitable condition to obtain racemic compound of formula (IV),

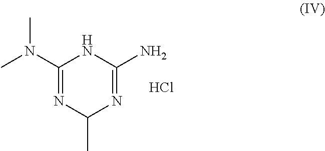

b) reacting the racemic compound of formula (IV) with a suitable chiral amino acid or its derivative in the presence of a base and a solvent to obtain R-Imeglimin L-amino acid salt compound of formula (V),

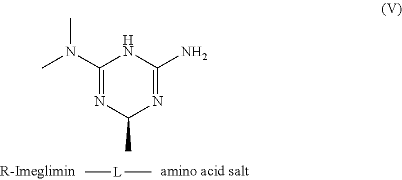

c) reacting the compound of formula (V) with hydrochloric acid in the presence of a solvent to obtain Imeglimin Hydrochloride formula (I), and
d) optionally purifying the Imeglimin Hydrochloride of formula (I) obtained in step (c) using an alcoholic solvent.

In another aspect, the invention provides a process for preparation of R-Imeglimin HCl compound of formula (I) via resolution of racemic Imeglimin base (IV) or Imeglimin HCl compound of formula (IV), which process comprises the steps of;
a) reacting racemic compound of formula (IV) or compound of formula (VI) with a suitable chiral amino acid or its derivative in the presence or absence of a base and in the presence of a solvent to obtain R-Imeglimin L-amino acid salt compound of formula (V),

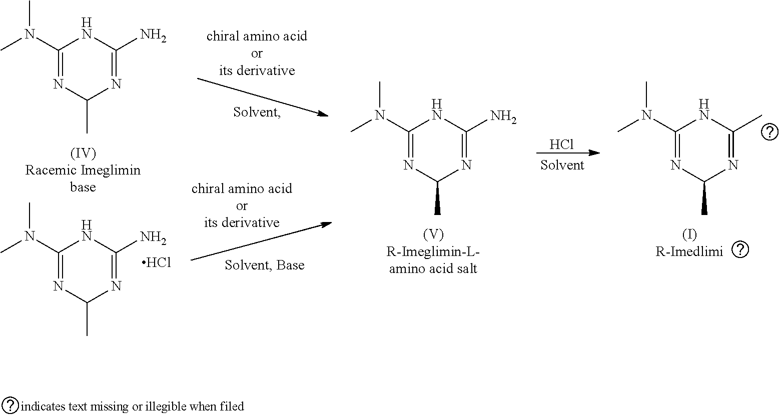

b) reacting the compound of formula (V) with hydrochloric acid in the presence of a solvent to obtain Imeglimin Hydrochloride of formula (I), and
c) optionally purifying the Imeglimin Hydrochloride of formula (I) obtained in step (b) using an alcoholic solvent.

### DETAILED DESCRIPTION OF THE INVENTION

In an attempt to develop an improved process for the preparation of Imeglimin HCl and to overcome the disadvantages of prior arts, the present inventors have developed a process which results in high chiral, HPLC purity and good yield of Imeglimin HCl.

The present invention provides a process for preparation of Imeglimin HCl compound of formula (I), which process comprises;
a) reacting compound of formula (II)

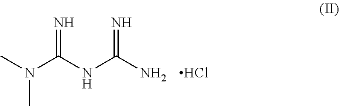

with compound of formula (III) in a solvent

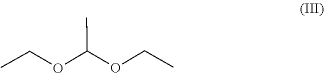

in presence of p-toluene sulphonic acid monohydrate under suitable condition to obtain racemic compound of formula (IV),

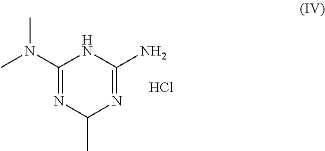

b) reacting the racemic compound of formula (IV) with a suitable chiral amino acid or its derivative in the presence of a base and a solvent to obtain R-Imeglimin L-amino acid salt compound of formula (V),

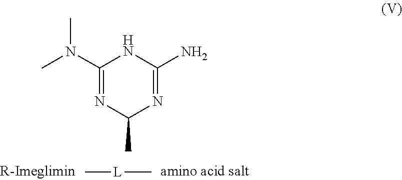

c) reacting the compound of formula (V) with hydrochloric acid in the presence of solvent to obtain Imeglimin Hydrochloride formula (I), and
d) optionally purifying the Imeglimin Hydrochloride of formula (I) obtained in step (c) using an alcoholic solvent.

In the embodiment of step, a), the solvent is selected from group consisting of C₁-C₄ alcohol, preferably, methanol, ethanol, isopropanol, isobutanol, water and mixtures thereof; more preferably, the solvent is isobutanol.

In the embodiment of step b), the chiral amino acid is selected from the group consisting of L-glutamic acid, N-Tosyl-L-glutamic acid, L-Alanine, L-aspartic acid, L-Leucine, L-phenyl alanine, L-tyrosine and, L-Valine.

In the embodiment of step b), the solvent is selected from group consisting of C₁-C₄ alcohol, preferably methanol, ethanol, isopropanol, isobutanol and mixtures thereof and the base is organic base such as triethylamine or inorganic base such as sodium hydroxide.

In the embodiment step c), the solvent is selected from group consisting of C₁-C₄ alcohol, preferably methanol, ethanol, isopropanol, isobutanol and mixtures thereof or C₁-C₅ ketone, preferably acetone, methyl ethyl ketone.

In the embodiment step d), the solvent is selected from group consisting of C₁-C₄ alcohol, preferably methanol, ethanol, isopropanol, isobutanol, water and mixtures thereof; more preferably, the solvent is mixture of methanol and isopropyl alcohol.

In one of the preferred embodiments, the chiral amino acid is selected from the group consisting of L-glutamic acid or its derivative such as N-tosyl-L-glutamic acid or N-sulfonyl-L-glutamic acid etc.

Accordingly, the process for preparation of Imeglimin HCl compound of formula (1) is demonstrated herein below in scheme III, using L-amino acid or its derivative as a chiral resolving agent.

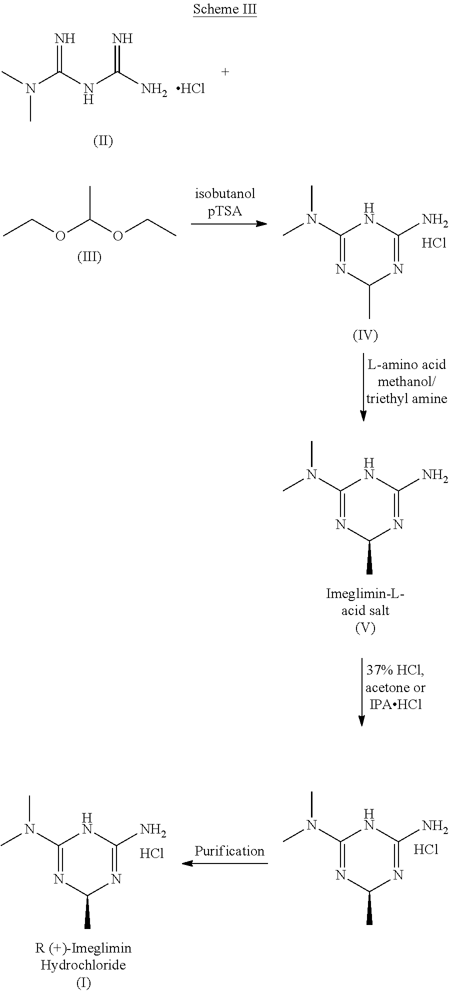

The starting material i.e. metformin hydrochloride, the compound of formula (II) & compound of formula (III) of the present invention are commercially available. Accordingly, in one of the embodiments, the present invention provides a process for preparation of Imeglimin HCl compound of formula (I)

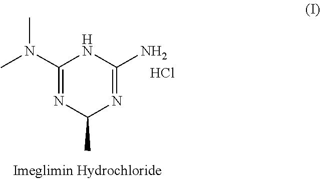

comprising steps of:
a) reacting compound of formula (II)

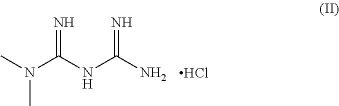

with compound of formula (III) in a solvent

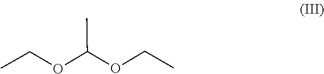

in presence of p-toluene sulphonic acid monohydrate under suitable condition to obtain racemic compound of formula (IV),

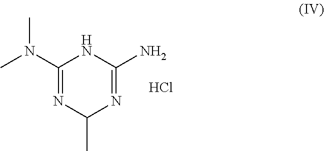

b) reacting the racemic compound of formula (IV) with L-Glutamic acid or its derivative in the presence of a base and a solvent to obtain compound of formula (V),

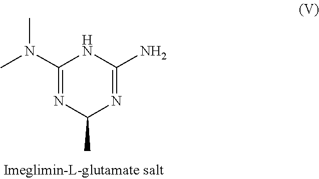

c) reacting the compound of formula (V) with hydrochloric acid in the presence of solvent to obtain Imeglimin Hydrochloride formula (I), and
d) optionally purifying the Imeglimin Hydrochloride of formula (I) obtained in step (c) using an alcoholic solvent.

In one of the preferred embodiments, the chiral amino acid is selected from the group consisting of L-glutamic acid or its derivative such as N-tosyl-L-glutamic acid or N-sulfonyl-L-glutamic acid etc.

Accordingly, the process for preparation of Imeglimin HCl compound of formula (I) is demonstrated herein below in scheme IV, using L-glutamic acid or its derivative as a chiral resolving agent.

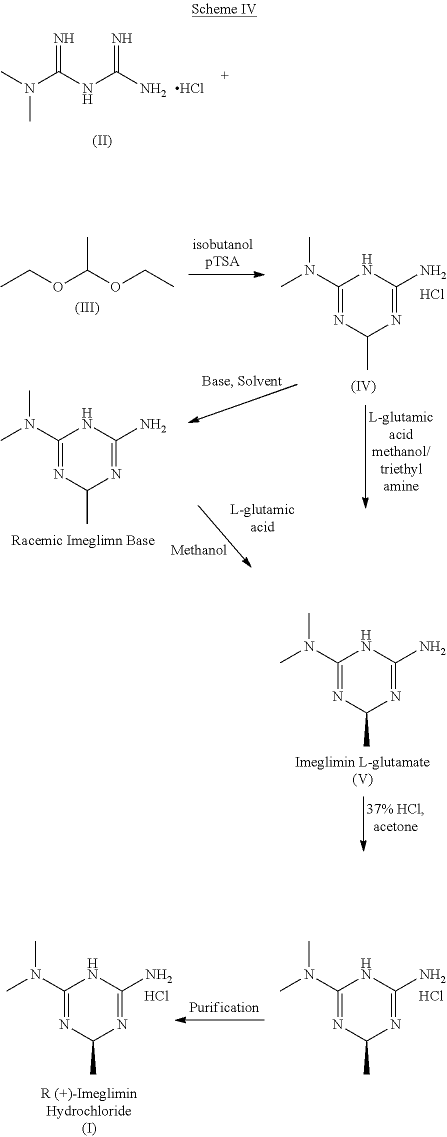

In yet another embodiment, the invention provides a process for preparation of Imeglimin HCl compound of formula (I) via resolution of racemic Imeglimin base compound of formula (IV) or racemic Imeglimin hydrochloride (VI), which process comprises the steps of;
a) reacting racemic compound of formula (IV) or compound of formula (VI) with a suitable chiral amino acid or its derivative in the presence or absence of a base and a solvent to obtain R-Imeglimin L-amino acid salt compound of formula (V),

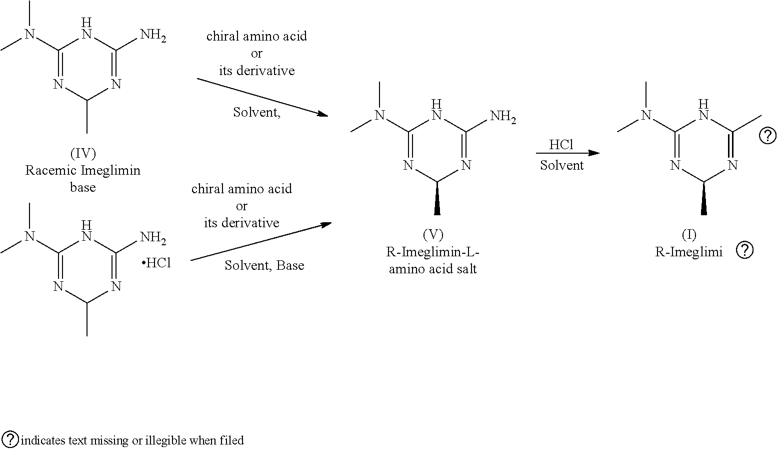

b) reacting the compound of formula (V) with hydrochloric acid in the presence of a solvent to obtain Imeglimin Hydrochloride formula (I), and
c) optionally purifying the Imeglimin Hydrochloride formula (I) obtained in step (b) using an alcoholic solvent.

In the embodiment of step a), the chiral amino acid is selected from the group consisting of L-glutamic acid, N-Tosyl-L-glutamic acid, L-Alanine, L-aspartic acid, L-Leucine, L-phenyl alanine, L-tyrosine and, L-Valine.

In the embodiment of step, a), the solvent is selected from group consisting of acetonitrile, C₁-C₄ alcohol, preferably methanol, ethanol, isopropanol, isobutanol and mixtures thereof.

In the embodiment step b), the solvent is selected from group consisting of C1-C4 alcohol, preferably methanol, ethanol, isopropanol, isobutanol and mixtures thereof or C1-C5 ketone, preferably acetone, methyl ethyl ketone.

In the embodiment step c), the solvent is selected from group consisting of C1-C4 alcohol, preferably methanol, ethanol, isopropanol, isobutanol, water and mixtures thereof; more preferably, the solvent is mixture of methanol and isopropyl alcohol.

Accordingly, the process for preparation of Imeglimin HCl compound of formula (I) via resolution of racemic Imeglimin base compound of formula (IV) or Imeglimin HCl compound of formula (VI), is demonstrated herein below in Scheme V, using L-glutamic acid or its derivative as a chiral resolving agent.

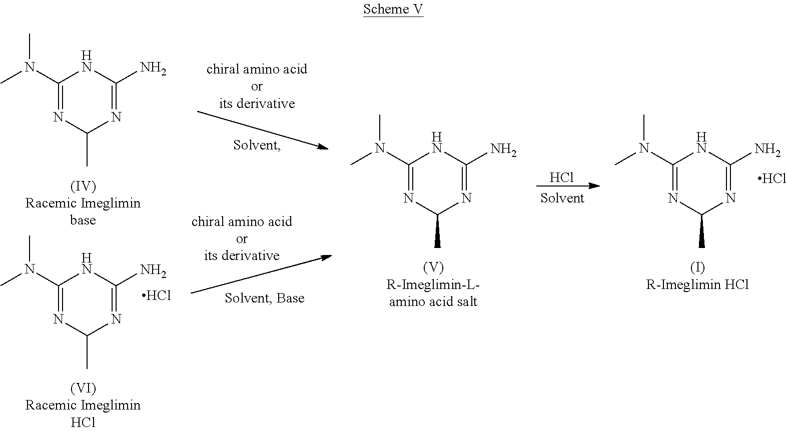

### EXAMPLES

The following examples are illustrative of some of the embodiments of the present invention described herein. These examples should not be considered to limit the spirit or scope of the invention in any way.

### Example 1: Synthesis of Racemic Imeglimin Hydrochloride

Metformin hydrochloride (100 g) was suspended into isobutanol (400 ml), acetaldehyde diethyl acetal (85.94 g) and para toluene sulfonic acid (11.52 g) were added to the suspension. The suspension was heated to reflux until a clear solution is obtained. After completion of the reaction solvent was partially distilled. The suspension was cooled to 10-20° C. & stirred for 2 hrs. The solid was isolated by filtration & washed with isobutanol (50 ml). The solid was dried to obtain racemic Imeglimin Hydrochloride (100 g).

### Example 2: Chiral Resolution of Racemic Imeglimin Hydrochloride to Obtain (R)-Imeglimin-L-Glutamate Salt

Racemic Imeglimin hydrochloride (20 g) & L-glutamic acid (15.4 g) were added in methanol (250 ml) at 20-35° C. Triethylamine (10.6 g) was added and the suspension was heated to 55-60° C. & stirred for 2 hrs. The suspension was cooled to room temperature is seeded with little quantity of R-Imeglimin L-glutamate salt and stirred for overnight to obtain solid. The reaction mass was cooled to 5-10° C. and stirred for 2 hrs. The white crystals obtained were isolated by filtration and washed with methanol (10 ml). Dried the solid to obtain R-(+)-Imeglimin-L-glutamate salt (15.3 gm).

Purity: HPLC 99.70%

### Example 3: Resolution of Racemic Imeglimin Hydrochloride

Racemic Imeglimin hydrochloride (20 g) & N-tosyl-L-glutamic acid (31.54 g) were dissolved in methanol (50 ml) at 20-35° C., triethyl amine (10.59 g) was added at below 30° C. The suspension was heated to 55-60° C. & stirred for 2.0 hrs. The suspension was cooled to RT. The suspension was seeded with R-Imeglimin N-tosyl-L-glutamate salt. The solution was stirred for 6 hrs, cooled the reaction mass to 5-10° C. for 2.0 hrs, to obtain solid. The white crystals thus obtained were isolated by filtration and washed with methanol (5 ml). The solid was dried to obtain R-Imeglimin N-tosyl-L-glutamate salt (20 g).

Purity: HPLC 99.95%

### Example 4: Resolution of Racemic Imeglimin Hydrochloride

Racemic Imeglimin hydrochloride (70 g) & N-Tosyl-L-glutamic acid (110.42 g) were added in Acetonitrile (700 ml) at 20-35° C. Triethyl amine (37.08 g) was added at below 30° C. The suspension was heated to 60-65° C. and water (35 ml) was added. The reaction mass was stirred for 2.0 hrs, cooled to RT. The white crystals were isolated by filtration and washed with Acetonitrile (35 ml), the wet cake was further purified in acetonitrile & water mixture. The solid thus obtained was dried to yield (82.6 g) R-Imeglimin N-tosyl-L-glutamate salt.

Purity: HPLC 99.95%

### Example 5: Preparation of R-Imeglimin Hydrochloride from (R)-Imeglimin N-Tosyl-L-Glutamate

Imeglimin N-tosyl-L-glutamate salt (80 g) was suspended in Acetone (400 ml) and IPA·HCl (84 g) was added. The suspension was heated to reflux and continued for 2.0 hrs, cooled the suspension to RT, stirred for 2 hrs. Filtered the solid & washed with Acetone (80 ml). The solid was dried to obtain crude (R) Imeglimin Hydrochloride (32.0 g).

Purity: HPLC 99.5%

### Example 6: Preparation of R-Imeglimin Hydrochloride from (R)-Imeglimin N-Tosyl-L-Glutamate

Imeglimin N-tosyl-L-glutamate salt (20 g) was suspended in Acetone (100 ml) added conc. HCl (4.8 g). The suspension was heated to reflux and continued for 2.0 hrs, cooled RT. Stirred for 2 hrs. Filtered the solid & washed with Acetone (80 ml). Dried the solid to obtain crude (R)-Imeglimin Hydrochloride (7.4 g).

Purity: HPLC 99.5%

### Example 7: Purification of (R) Imeglimin Hydrochloride

Crude (R)-Imeglimin hydrochloride (10.0 g) was dissolved in methanol (20 ml) at 55-60° C. Filtered the clear solution & distilled out solvent to obtain suspension. Added IPA (50 ml) and heated to 55-60° C. Stirred for 1.0 hr & cooled to RT. Filtered the solid & washed with IPA (10 ml). Dried the solid to obtain pure (R) Imeglimin hydrochloride (8.5 g).

Purity: HPLC 99.99%, Specific Optical Rotation=+5.3°

### Example-8: Preparation of Racemic Imeglimin Base from Imeglimin Hydrochloride

Racemic Imeglimin Hydrochloride (25 g) was reacted with sodium methoxide (7.04) g in methanol (100 ml) at room temperature. Salt was removed by filtration. Methanol was collected as filtrate and was distilled out to obtain Imeglimin base (19 g).

### Example-9: Resolution of Racemic Imeglimin Base

Racemic Imeglimin (18 g) & N-Tosyl-L-glutamic acid (34.87 g) were added in Acetonitrile (90 ml) at 50-55° C. Stirred for 2 hours, the white crystals were isolated by filtration and washed with Acetonitrile (18 ml). The solid thus obtained was dried to yield R-Imeglimin N-tosyl-L-glutamate salt (22.0 g).

### Example 10: Preparation of R-Imeglimin Hydrochloride from (R)-Imeglimin N-Tosyl-L-Glutamate

Imeglimin N-tosyl-L-glutamate salt (20 g) obtained from Example-9 was suspended in Acetone (100 ml) and IPA·HCl (21.0 g) was added. The suspension was heated to reflux and continued for 2.0 hrs, cooled the suspension to RT, stirred for 2 hrs. Filtered the solid & washed with Acetone (20 ml). The solid was dried to obtain crude (R) Imeglimin Hydrochloride (8.0 g).

Purity: HPLC 99.5%

1. A process for the preparation of an imeglimin HCl compound of formula (I), which process comprises:
a) reacting a compound of formula (II) 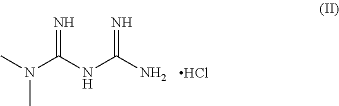
with a compound of formula (III) in a solvent 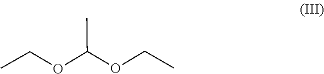
in the presence of p-toluene sulphonic acid monohydrate under a suitable condition to obtain a racemic compound of formula (IV), 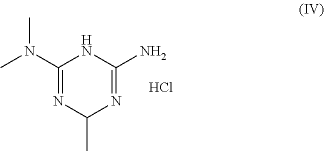
b) reacting the racemic compound of formula (IV) with a suitable chiral amino acid or its derivative in the presence of a base and a solvent to obtain R-imeglimin L-amino acid salt compound of formula (V) 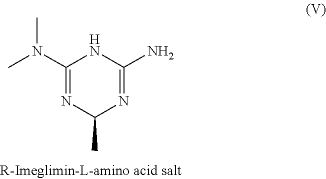
c) reacting the compound of formula (V) with hydrochloric acid in the presence of a solvent to obtain imeglimin hydrochloride of formula (I) 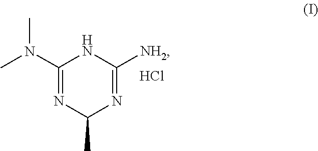
and
d) optionally purifying the imeglimin hydrochloride of formula (I) obtained in step (c) using an alcoholic solvent.

2. A process for the preparation of an imeglimin HCl compound of formula (I) via resolution of a racemic imeglimin base or an imeglimin HCl, which process comprises the steps of:
a) reacting a racemic compound of formula (IV) or a compound of formula (VI) with a suitable chiral amino acid or its derivative in the presence or absence of a base and in the presence of a solvent to obtain R-imeglimin L-amino acid salt compound of formula (V), 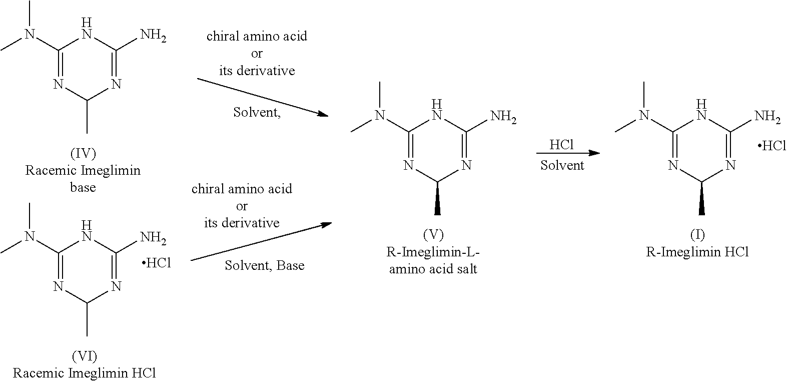
b) reacting the compound of formula (V) with hydrochloric acid in the presence of a solvent to obtain imeglimin hydrochloride of formula (I), and
c) optionally purifying the imeglimin hydrochloride of formula (I) obtained in step (b) using an alcoholic solvent.

3. The process as claimed in claim 1, wherein the solvent used in step a) is selected from the group consisting of C₁-C₄ alcohol, preferably methanol, ethanol, isopropanol, isobutanol, water and a mixture thereof.

4. The process as claimed in claim 1, wherein, the chiral amino acid is selected from the group consisting of L-glutamic acid, N-tosyl-L-glutamic acid, L-alanine, L-aspartic acid, L-leucine, L-phenyl alanine, L-tyrosine and, and L-valine.

5. The process as claimed in claim 1, wherein the solvent used in the chiral resolution step is selected from the group consisting of acetonitrile, a C₁-C₄ alcohol, and a mixture thereof.

6. The process as claimed in claim 1, wherein the solvent used in the reaction of the compound of formula (V) with hydrochloric acid is selected from the group consisting of a C₁-C₄ alcohol, and a C₁-C₅ ketone, preferably acetone or methyl ethyl ketone.

7. The process as claimed in claim 1, wherein the solvent used in the purification of the imeglimin hydrochloride of formula (I) is selected from the group consisting of a C₁-C₄ alcohol, water, and a mixture thereof.

8. The process as claimed in claim 1, wherein the chiral amino acid is L-glutamic acid or its derivative.

9. The process as claimed in claim 8, wherein the derivative of L-glutamic acid is selected from the group consisting of N-tosyl-L-glutamic acid and N-sulfonyl-L-glutamic acid.

10. The process for the preparation of the imeglimin HCl compound of formula (I), wherein the process comprising the steps of: 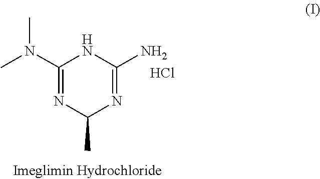
a) reacting a compound of formula (II) 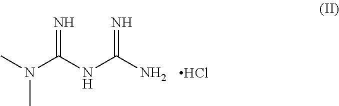
with a compound of formula (III) in a solvent 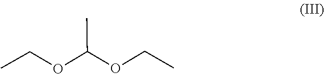
in the presence of p-toluene sulphonic acid monohydrate under a suitable condition to obtain a racemic compound of formula (IV), 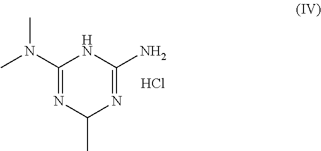
b) reacting the racemic compound of formula (IV) with L-glutamic acid or its derivative in the presence of a base and a solvent to obtain a compound of formula (V); and 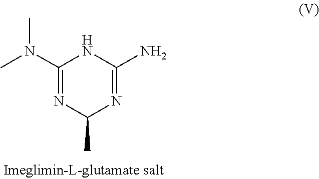
c) reacting the compound of formula (V) with hydrochloric acid in the presence of a solvent to obtain imeglimin hydrochloride of formula (I), and
d) optionally purifying the imeglimin hydrochloride of formula (I) obtained in step (c) using an alcoholic solvent.

11. The process as claimed in claim 10, wherein; the derivative of L-glutamic acid is selected from the group consisting of N-tosyl-L-glutamic acid and N-sulfonyl-L-glutamic acid.

12. The process as claimed in claim 2, wherein the chiral amino acid is selected from L-glutamic acid, N-tosyl-L-glutamic acid, L-alanine, L-aspartic acid, L-leucine, L-phenyl alanine, L-tyrosine and, and L-valine.

13. The process as claimed in claim 2, wherein the solvent used in the chiral resolution step is selected from the group consisting of acetonitrile, a C₁-C₄ alcohol, and a mixture thereof.

14. The process as claimed in claim 2, wherein the solvent used in the reaction of the compound of formula (V) with hydrochloric acid is selected from the group consisting of a C₁-C₄ alcohol and a C₁-C₅ ketone.

15. The process as claimed in claim 2, wherein the solvent used in the purification of the imeglimin hydrochloride of formula (I) is selected from the group consisting of a C₁-C₄ alcohol, water, and a mixture thereof.

16. The process as claimed in claim 2, wherein the solvent used in the chiral resolution step is methanol, ethanol, isopropanol, isobutanol or a mixture thereof; the solvent used in the reaction of the compound of formula (V) with hydrochloric acid is selected from the group consisting of methanol, ethanol, isopropanol, isobutanol, acetone, methyl ethyl ketone, and a mixture thereof; and the solvent used in the purification of the imeglimin hydrochloride of formula (I) is selected from the group consisting of methanol, ethanol, isopropanol, isobutanol, water, and a mixture thereof.

17. The process as claimed in claim 2, wherein the chiral amino acid is L-glutamic acid or its derivative.

18. The process as claimed in claim 17, wherein the derivative of L-glutamic acid is selected from the group consisting of N-tosyl-L-glutamic acid and N-sulfonyl-L-glutamic acid.

19. The process as claimed in claim 1, wherein the solvent used in the chiral resolution step is methanol, ethanol, isopropanol, isobutanol or a mixture thereof; the solvent used in the reaction of the compound of formula (V) with hydrochloric acid is selected from the group consisting of methanol, ethanol, isopropanol, isobutanol, acetone, methyl ethyl ketone, and a mixture thereof; and the solvent used in the purification of the imeglimin hydrochloride of formula (I) is selected from the group consisting of methanol, ethanol, isopropanol, isobutanol, water, and a mixture thereof.
